(19) United States
(12) Patent Application Publication
(10) Pub. No.: US 2023/0026072 A1
(43) Pub. Date: Jan. 26, 2023

(54) SYSTEM AND METHOD FOR LOW LATENCY MOTION INTENTION DETECTION USING SURFACE ELECTROMYOGRAM SIGNALS

(71) Applicant: BRINK BIONICS INC., Waterloo (CA)

(72) Inventors: Erik LLOYD, Waterloo (CA); Ning JIANG, Waterloo (CA); Jiayuan HE, Waterloo (CA)

(21) Appl. No.: 17/788,792

(22) PCT Filed: Dec. 3, 2020
(86) PCT No.: PCT/CA2020/051662

§ 371 Date: Jun. 24, 2022

Related U.S. Application Data
(60) Provisional application No. 62/953,447, filed on Dec. 24, 2019.

(51) Int. Cl. A61B 5/395; G06F 3/01

(52) U.S. Cl. CPC A61B 5/395; G06F 3/015

(57) ABSTRACT

System for detecting intention of movements by a subject comprises sensors and a computing device. The sensors are configured to be engaged to the subject. The sensors measure electromyogram signals from the subject. The computing device receives the electromyogram signals from the sensors. Features are extracted using electromyogram signals from one or more of the sensors. One or more of the extracted features are compared with their respective threshold corresponding to a first movement among the movements. Intention of making the first movement is registered, prior to the onset of the first movement, based on the comparison.

### BACKGROUND

Unless otherwise indicated herein, the materials described in this section are not prior art to the claims in this application and are not admitted to being prior art by inclusion in this section.

### FIELD

The subject matter in general relates to human-machine interaction. More particularly, but not exclusively, the subject matter relates to detection of intention of human motions using surface electromyogram (sEMG) signals.

### DISCUSSION OF THE RELATED ART

Mechanical movements in humans and animals are driven by the contraction of skeletal muscles, and the muscle contraction is accompanied by a series of inherent electrical activities in muscle fibers. The electrical activities may be measured by attaching electrodes (sensors) on skin above these muscles. The collected signal may be called surface electromyogram (sEMG) signal or myoelectric signal. There may be time lag, electromechanical delay (EMD), between the onset of the electrical process and the onset of mechanical movement of humans. The motion intension can be detected before the corresponding overt mechanical movement because of EMD. The detection of sEMG signal may be utilised to understand an individual's intention to move before overt mechanical movement is pre-set, resulting in enhancing the performance of human-machine and human-computer interaction. As an example, a gamer may send commands in PC gaming using a keyboard and a mouse, which is controlled by the mechanical movement of gamer's hands. If the command is sent from the onset of the electrical process of muscle contraction, it could be sent earlier to the PC, and the players may get the corresponding response earlier, which increases chance to win in gaming. Other applications include the control of exoskeleton and robotics, among others.

The EMD value is typically around 50 ms, varying depending on several factors, including muscle type, age and gender, among others. To achieve the detection of motion intention before the onset of overt mechanical movement, the latency from the sEMG signal processing must be kept low, and smaller than the EMD value. Currently, the dominant application of myoelectric control is to help amputees control prosthetic hand. As the requirement of the delay in prothetic control is around 300 ms, much larger than EMD, conventional technologies fall short in achieving detection of motion intention earlier than the mechanical movement.

In view of the foregoing discussion, there is a need for improved technical solution for detection of intention of human motion using sEMG signals.

### SUMMARY

In an aspect, a system is provided for detecting intention of movements by a subject. The system comprises sensors and a computing device. The sensors are configured to be engaged to the subject. The sensors measure electromyogram signals from the subject, which are received by the computing device. Features are extracted using electromyogram signals from one or more of the sensors. One or more of the extracted features are compared with their respective threshold corresponding to a first movement among the movements. Intention of making the first movement is registered, prior to the onset of the first movement, based on the comparison.

### BRIEF DESCRIPTION OF DIAGRAMS

This disclosure is illustrated by way of example and not limitation in the accompanying figures. Elements illustrated in the figures are not necessarily drawn to scale, in which like references indicate similar elements and in which:

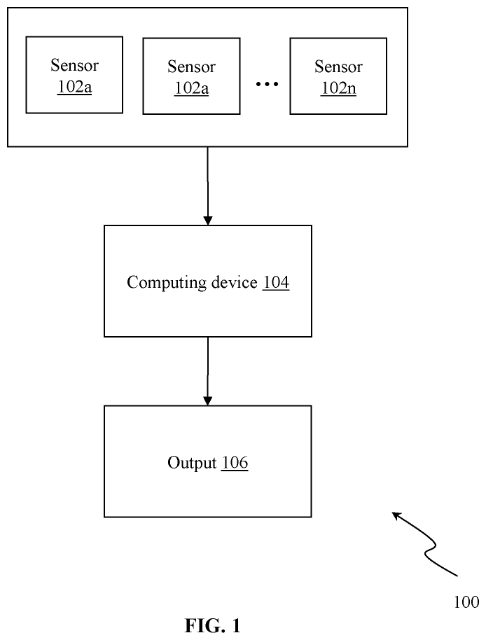

FIG. 1 illustrates a system 100 for low latency motion intention detection using surface electromyogram signals, in accordance with an embodiment;

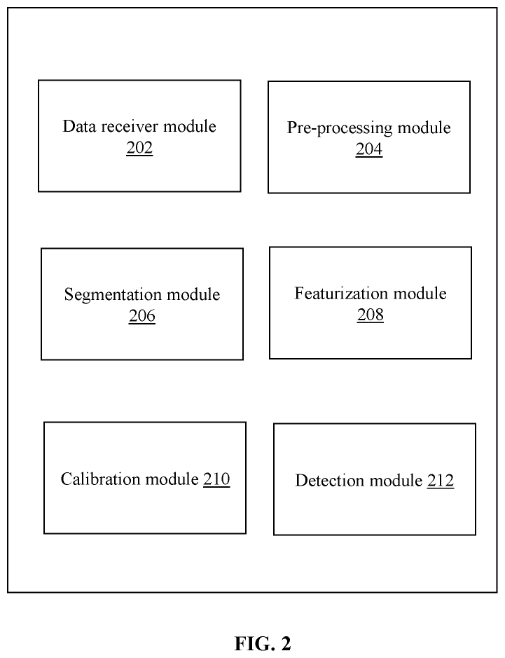

FIG. 2 illustrates various modules of a computing device 104 of the system 100, in accordance with an embodiment;

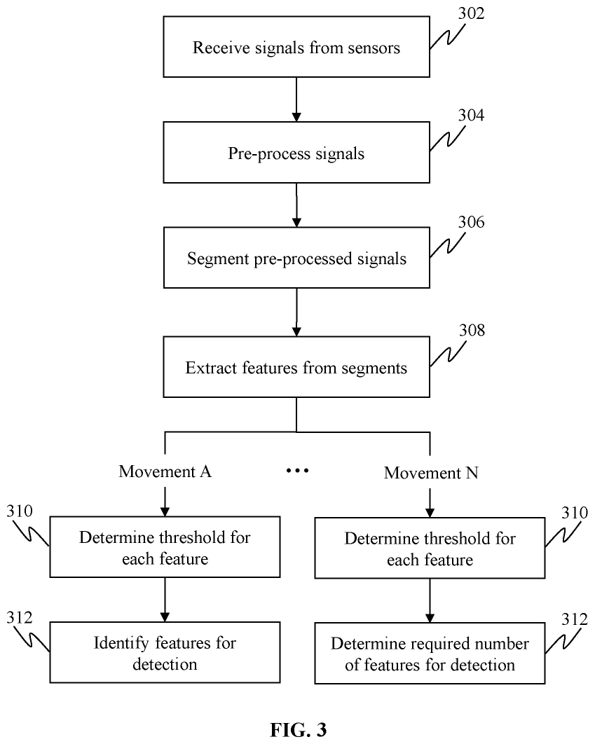

FIG. 3 is a flowchart illustrating the method of calibrating the system 100, in accordance with an embodiment;

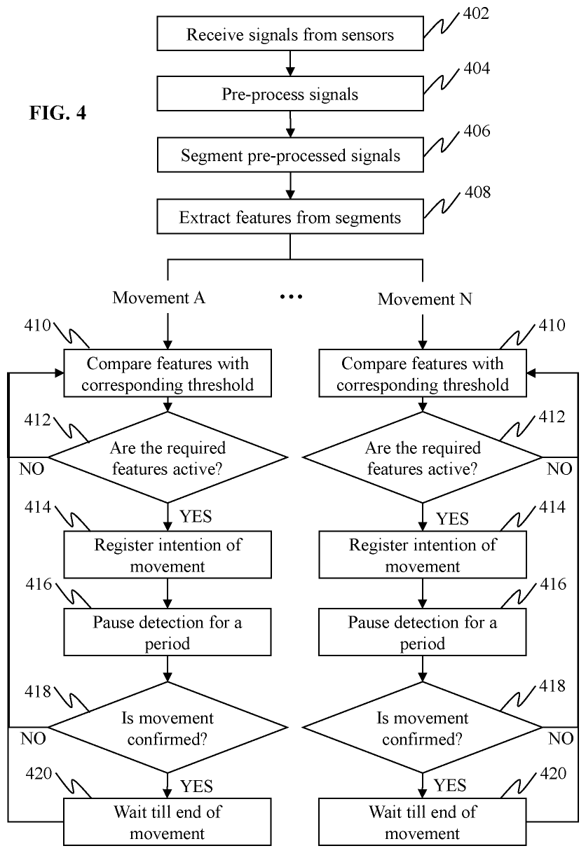

FIG. 4 is a flowchart illustrating the method of detecting intention of a mechanical movement in real-time, in accordance with an embodiment; and

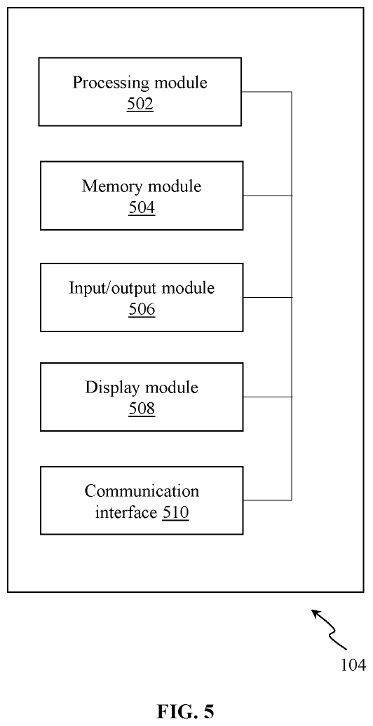

FIG. 5 illustrate a hardware configuration of the computing device 104, in accordance with an embodiment.

### DETAILED DESCRIPTION OF THE INVENTION

The following detailed description includes references to the accompanying drawings, which form part of the detailed description. The drawings show illustrations in accordance with example embodiments. These example embodiments are described in enough detail to enable those skilled in the art to practice the present subject matter. However, it may be apparent to one with ordinary skill in the art that the present invention may be practised without these specific details. In other instances, well-known methods, procedures and components have not been described in detail so as not to unnecessarily obscure aspects of the embodiments. The embodiments can be combined, other embodiments can be utilized, or structural and logical changes can be made without departing from the scope of the invention. The following detailed description is, therefore, not to be taken in a limiting sense.

In this document, the terms “a” or “an” are used, as is common in patent documents, to include one or more than one. In this document, the term “or” is used to refer to a non-exclusive “or”, such that “A or B” includes “A but not B”, “B but not A”, and “A and B”, unless otherwise indicated.

Referring to the figures, and more particularly to FIG. 1, system 100 and method for detecting an intention of motion of a subject are discussed. The system 100 detects the intention of motion with low latency by using surface electromyogram (sEMG) signals.

The system 100 may comprise a plurality of sensors 102a, 102b . . . 102n (may be referred to as sensor 102 or sensors 102), a computing device 104 and an output 106. The sensors 102 may be configured to be attached on the skin above the muscles of a subject, such as a human. Mechanical movement of the subject, which is driven by the contraction of skeletal muscles, is accompanied by a series of inherent electrical activities in muscle fibers, which may be measured by the sensors 102. The collected signals may be called surface electromyogram (sEMG) signal, or myoelectric signal.

The signals measured by the sensors 102 may be sent to the computing device 104 for processing. Examples of computing device 104 include, but not limited to, smart phone, tablet PC, notebook PC, desktop, gaming device, robotic system, workstation or laptop, among like computing devices. The computing device 104 may be configured to process and analyse the signals to determine the intention of the movement of the subject before the corresponding overt mechanical movement. As an example, consider a set of sensors 102 attached to the arm of a person. The sensors 102 may measure the signals generated and may communicate the signals to the computing device 104. The computing device 104 may process and analyse to arrive at a conclusion that the signals generated are a pre-cursor to a particular movement of the arm. Such a determination may be output by the computing device 104 to the output 106, such as, for example, computer, a cell phone, tablet, gaming console or other like devices.

The system 100 may be calibrated for enabling determination of the intention of the movement in real-time. Examples of movements include, but not limited to various types of, finger movements, wrist movements and joint movements. The system 100 may be calibrated for a variety of movements. Once the system 100 is calibrated, in real-time, the signals may be processed based on the calibration to determine motion intention at low latency. Referring to FIGS. 2 and 3, calibration of the system 100 is discussed. The computing device 104 may comprise a data receiver module 202, a pre-processing module 204, segmentation module 206, featurization module 208, calibration module 210 and a detection module 212.

At step 302, the signal from the sensors 102 may be received by the data receiver module 202. As discussed earlier, muscle contracts as a pre-cursor to overt mechanical movement, and electric signal are generated that may propagate through adjacent tissue and may be recorded at neighbouring skin area. The sensors 102 attached over the skin area measure the signals and communicate the signals to the data receiver module 202.

At step 304, the pre-processing module 204 may process the received signals. In an embodiment, the pre-processing module 204 may lowpass filter the signals by an anti-aliasing filter to remove motion artifacts. The low frequency range may be between 5 Hz and 30 Hz. The signals may be further, optionally, notch filtered (50 Hz or 60 Hz, as an example, based on the local line frequency) to reject the main inference. The filtered signals may be digitized. The digitized signals may be sent to the segmentation module 208.

At step 306, the segmentation module 208 may segment the digitized signals to enable features extraction from the segments. Each of segments may be in milliseconds or tens of milliseconds depending on the specific feature extraction technique used. Preferably, each of the segments may be less than 50 ms. As discussed earlier, typically, the EMD is around 50 ms. Hence the segment length may be set to a value less than 50 ms to avoid the long latency. There may be overlap between the two consecutive segments.

At step 308, the segmented signals may be sent to the featurization module 208 for extracting features from the segments. The features may be extracted from the signal segments, either from a single sensor 102 or multiple sensors 102, to represent the energy or energy change-related information. As an example, suppose x(n) is a sample of digital data from one sensor 102, where “n” is its sequence, x(n−1) is its predecessor, and x(n+1) is its successor, y(n) and z(n) are two samples of digital signals from neighbouring sensors 102, respectively. The features extracted may include, but not limited to the following:

x²(n)−x(n+1)×x(n−1) Feature 1:

x⁶(n)−x³(n+1)×x³(n−1) Feature 2:

x²(n)−y(n)×z(n) Feature 3:

x²(n)×y(n)×z(n) Feature 4:

x²(n)×x(n+1)×x(n−1) Feature 5:

Example of another feature extracted may be the root mean square of the three consecutive samples from one physical sensor.

Yet another example of feature extracted may be average of the three consecutive samples from one physical sensor.

It should be noted that other features representing the energy or energy change of the data, such as the mean, the root mean square, could also be used as features.

At step 310, the calibration module 210 may determine threshold values for different features for a variety of movements for which calibration is carried out. In an embodiment, to determine the threshold values for different features corresponding to a specific movement, the duration of the mechanical movement may be divided into four periods, such as, pre-motion, motion-execution, after-motion, and rest. Pre-Motion may be a period before each onset of the mechanical movement, which may last as long as 200 ms. Motion-execution may be period from each onset of the mechanical movement to its end. After-motion may be a period after each end of the mechanical movement, which may last as long as 200 ms. The remaining part may be defined as Rest. Threshold value of a specific feature, e.g., Feature-A, for a specific mechanical movement may be the maximum value of Feature-A over the pre-motion period. In other words, in real-time detection of this specific mechanical movement, for one incident of this movement, if there is a Feature-A sample in the pre-motion whose value is equal to or larger than the corresponding threshold, this movement may be detected by Feature-A.

At step 312, the calibration module 210 may determine required features for detection of a particular mechanical movement. As one may appreciate, since there may be set of features under consideration, multiple features in that set may be able to indicate intention of a specific mechanical movement. Hence, the calibration module 210 identifies one or more features (may be referred as selected features) in the set for successfully detecting a specific mechanical movement. The selected features may be obtained through an exhaustive search considering individual and combination of features in the set. In each search, two metrics may be calculated, i.e. the detection accuracy and averaged time lead. Lead time of a feature may be the duration between the time at which the feature has a value equal to or larger than its threshold and the onset of the specific mechanical movement. When comparing the performance, the detection accuracy may be given priority. If there are multiple scenarios achieving the same detection accuracy, the averaged time lead may be used to determine the required features for the specific mechanical movement.

Having discussed the method for calibrating the system, the method for detection of intention of a mechanical movement in real-time is discussed hereunder.

FIG. 4 is a flowchart illustrating the steps involved in the detection of intention of a mechanical movement in real-time, in accordance with an embodiment. The steps may be executed by a computing device such as the one discussed earlier. Such a device may not necessarily have a calibration module 210, instead have the calibration values to enable such detection.

At step 402, the signal from the sensors 102 may be received by the data receiver module 202. The sensors 102 attached over the skin area measure the signals and communicate the signals to the data receiver module 202.

At step 404, the pre-processing module 204 may process the received signals. In an embodiment, the pre-processing module 204 may lowpass filter the signals by an anti-aliasing filter to remove motion artifacts. The low frequency range may be between 5 Hz and 30 Hz. The signal may further, optionally, notch filtered (50 Hz or 60 Hz, as an example, based on the local line frequency) to reject the main inference. The filtered signals may be digitized. The digitized signals may be sent to the segmentation module 208.

At step 406, the segmentation module 208 may segment the digitized signals to enable features extraction from the segments. Each of segments may be in milliseconds or tens of milliseconds depending on the specific feature extraction technique used. Preferably, each of the segments may be less than 50 ms.

At step 408, the segmented signals may be sent to the featurization module 208 for extracting features from the segments. The features may be extracted from the segments, either from a single sensor 102 or multiple sensors 102 as discussed earlier in relation to step 308.

In an embodiment, the detection of each type of mechanical movement works in parallel. In other words, the detection of each type of mechanical movement may be performed simultaneously and independently of each other.

At step 410, features selected during calibration for a specific movement (e.g., movement A) may be compared, by the detection module 212, with their respective threshold set for the specific movement during calibration. In case the feature being compared meets the threshold requirement, then that feature may be labelled as being in an active state and comparison with the threshold may be paused until it is set back to an inactive state. The feature may be retained in the active state for a predefined period, which may be referred as intention detection period, which may be less than or equal to 200 ms. The intention detection period may be changed manually using a digital user interface. Likewise, the threshold of a specific feature for specific movement may be changed manually using a digital user interface.

In an embodiment, the thresholds of the features could be scaled synchronously with the same proportion, for example by the user, to adapt to the behaviour change during use.

At step 412, the detection module 212, may verify whether the required features are active at a given instance. It may be recollected that, during calibration, one or more features may be selected as required features to detect intention, of motion for a specific movement type, with a desired level of performance. Consequently, during detection of intention of a specific movement one or more features may be required to be in the active state at an instance for the intention of movement to be registered.

At step 414, the detection module 212, may register intention of the user to make the specific movement if the required features are in active state.

At step 416, the detection module 212, after registering the intention, may pause detection of this movement for a predefine period, which may be less than or equal to 100 ms.

At step 418, the detection module 212, after pausing the detection of intention of making the specific movement, verifies whether the corresponding movement did happen or not. As an example, a mechanical movement of an index figure to click a mouse may be detected by a mouse click, and thereby indicating to the system whether the mechanical movement happened or not.

In case the detection module 212 determines that the movement, whose intention was registered, did not occur, then the detection module 212 thereafter resumes the steps for detecting the intention of that movement.

On the other hand, if the detection module 212 determines that the movement, whose intention was registered, occurred, then the detection module 212 resumes the steps for detecting the intention of that movement only after that movement is completed (step 420).

FIG. 5 illustrates a hardware configuration of the computing device 104, in accordance with an embodiment.

In an embodiment, the computing device 104 may include one or more processors 502. The processor 502 may be implemented as appropriate in hardware, computer-executable instructions, firmware, or combinations thereof. Computer-executable instruction or firmware implementations of the processor 502 may include computer-executable or machine-executable instructions written in any suitable programming language to perform the various functions described. Further, the processor 502 may execute instructions, provided by the various modules of the computing device 104.

In an embodiment, the computing device 104 may include a memory module 504. The memory module 504 may store additional data and program instructions that are loadable and executable on the processor 502, as well as data generated during the execution of these programs. Further, the memory module 504 may be volatile memory, such as random-access memory and/or a disk drive, or non-volatile memory. The memory module 504 may be removable memory such as a Compact Flash card, Memory Stick, Smart Media, Multimedia Card, Secure Digital memory, or any other memory storage that exists currently or will exist in the future.

In an embodiment, the computing device 104 may include an input/output module 506. The input/output module 506 may provide an interface for inputting devices such as keypad, touch screen, mouse, and stylus among other input devices; and output devices such as speakers, printer, and additional displays among others.

In an embodiment, the computing device 104 may include a display module 508. The display module 508 may also be used to receive an input from a user. The display module 508 may be of any display type known in the art, for example, Liquid Crystal Displays (LCD), Light emitting diode displays (LED), Orthogonal Liquid Crystal Displays (OLCD) or any other type of display currently existing or may exist in the future.

In an embodiment, the computing device 104 may include a communication interface 510. The communication interface 510 may provide an interface between the computing device 104 and external networks. The communication interface 510 may include a modem, a network interface card (such as Ethernet card), a communication port, or a Personal Computer Memory Card International Association (PCMCIA) slot, among others. The communication interface 410 may include devices supporting both wired and wireless protocols.

The processes described above is described as a sequence of steps, this was done solely for the sake of illustration. Accordingly, it is contemplated that some steps may be added, some steps may be omitted, the order of the steps may be re-arranged, or some steps may be performed simultaneously.

The example embodiments described herein may be implemented in an operating environment comprising software installed on a computer, in hardware, or in a combination of software and hardware.

Although embodiments have been described with reference to specific example embodiments, it will be evident that various modifications and changes may be made to these embodiments without departing from the broader spirit and scope of the system and method described herein. Accordingly, the specification and drawings are to be regarded in an illustrative rather than a restrictive sense.

Many alterations and modifications of the present invention will no doubt become apparent to a person of ordinary skill in the art after having read the foregoing description. It is to be understood that the phraseology or terminology employed herein is for the purpose of description and not of limitation. It is to be understood that the description above contains many specifications, these should not be construed as limiting the scope of the invention but as merely providing illustrations of some of the personally preferred embodiments of this invention.

What is claimed is:

1. A system for detecting intention of movements by a subject, the system comprising:
a plurality of sensors configured to be engaged to the subject, the sensors configured to measure electromyogram signals from the subject; and
a computing device comprising at least one processor configured to:
receive the electromyogram signals from the sensors;
extract features using electromyogram signals from one or more of the sensors;
compare one or more of the extracted features with their respective threshold corresponding to a first movement among the movements; and
register intention of making the first movement, prior to the onset of the first movement, based on the comparison.

2. The system of claim 1, wherein the at least one processor is configured to compare multiple features, among the extracted features, with their respective threshold corresponding to the first movement, to decide whether the there is intention of making the first movement.

3. The system of claim 2, wherein the at least one processor is configured to register the intention of making the first movement if each of the multiple features meets their respective threshold corresponding to the first movement.

4. The system of claim 3, wherein the at least one processor is configured to:
set a first of the multiple features to an active state for an intention detection period after the first of the multiple features meets its respective threshold; detect whether remaining of the multiple features meet their respective threshold within the intention detection period;
register the intention of making the first movement if each of the multiple features meets their respective threshold within the intention detection period; and
reset the first of the multiple features to an inactive state, if each of the multiple features fails to meet their respective threshold within the intention detection period.

5. The system of claim 4, wherein the intention detection period is configured to be changed manually using a digital user interface.

6. The system of claim 2, wherein the multiple features are selected for the first movement based on accuracy of detection of the first movement during calibration of the system.

7. The system of claim 6, wherein the multiple features are selected for the first movement based on averaged lead time of the multiple features, wherein the lead time of each of the multiple features is a duration between the feature meeting its respective threshold and onset of the first movement.

8. The system of claim 7, wherein the accuracy of detection takes priority over the averaged lead time.

9. The system of claim 2, wherein the multiple features are selected for the first movement during calibration of the system, by considering individual and combination of the features extracted using the electromyogram signals from the sensors.

10. The system of claim 2, wherein the multiple features are selected for the first movement during calibration of the system, by considering individual and combination of the features extracted using the electromyogram signals from the sensors.

11. The system of claim 1, wherein the threshold of a first of the extracted features, corresponding to the first movement, is different compared to the threshold of the first of the extracted features, corresponding to a second movement among the movements.

12. The system of claim 1, wherein one or more of the extracted features represent energy or energy change-related information.

13. The system of claim 1, wherein the at least one processor is configured to detect intention of two or more of the movements simultaneously and independently of each other.

14. The system of claim 1, wherein the extracted features comprise one or more of:
square of one sample from a first sensor minus product between a predecessor sample and a successor sample of the first sensor;
square of the one sample from the first sensor minus product between one sample each from two neighboring sensors;
sixth power of the one sample from the first sensor minus product between third power of the predecessor sample and the third power of the successor sample;
product between square of the one sample from the first sensor and the product of the predecessor sample and the successor sample;
product between square of the one sample from the first sensor and product of the one sample each of the two neighboring sensors;
root mean square of three consecutive samples from the first sensor; or average of the three consecutive samples from the first sensor.

15. The system of claim 1, wherein the at least one processor is configured to pause the detection of intention of making the first movement, for a predefined period, after registering the intention of making the first movement.

16. The system of claim 15, wherein the predefined period is less than or equal to 100 ms.

17. The system of claim 15, wherein the at least one processor is configured to resume determination of the intention of making the first movement, after the pause, if the first movement fails to occur during the predefined period.

18. The system of claim 15, wherein the at least one processor is configured to resume determination of the intention of making the first movement after completion of the first movement, if the first movement occurs during the predefined period.

19. The system of claim 1, wherein the threshold of each of the one or more of the extracted features is a maximum value of the threshold corresponding to the first movement prior to onset of the first movement.

20. The system of claim 1, wherein the threshold of one or more of the features corresponding to respective movements is configured to be changed manually using a digital user interface.
